(19) United States
(12) Patent Application Publication
(10) Pub. No.: US 2022/0087901 A1
(43) Pub. Date: Mar. 24, 2022

(54) SYSTEMS AND APPROACHES FOR DRUG DELIVERY DEVICE RECONSTITUTION

(71) Applicant: AMGEN INC., Thousand Oaks, CA (US)

(72) Inventors: Nicholas J. Clark, Thousand Oaks, CA (US); Chia-Jung Wu, Redondo Beach, CA (US); Mingda Eu, Oak Park, CA (US); Artemio A. Alanis, Danville, CA (US); Jennifer Lyn Sawicky, Thousand Oaks, CA (US); Monica Goss, Newbury Park, CA (US); Jennifer Stevenson, Newbury Park, CA (US); Heather N. Franey, Ventura, CA (US); Deirdre Piedmonte, Agoura Hills, CA (US); Stephen Robert Brych, Thousand Oaks, CA (US)

(21) Appl. No.: 17/425,412

(22) PCT Filed: Feb. 12, 2020
(86) PCT No.: PCT/US2020/017874

§ 371 Date: Jul. 23, 2021

Related U.S. Application Data
(60) Provisional application No. 62/877,286, filed on Jul. 22, 2019.
(60) Provisional application No. 62/804,478, filed on Feb. 12, 2019.

(51) Int. Cl. A61J 1/20; A61M 39/00

(52) U.S. Cl. CPC A61J 1/2089; A61M 2039/0027; A61M 39/00; A61J 1/2096

(57) ABSTRACT

An approach for preparing a drug delivery device includes providing a prefilled delivery container and transferring a drug product contained in a drug container to the prefilled delivery container. The prefilled delivery container includes a container body containing a predetermined quantity of saline solution and a predetermined quantity of IV stabilizing solution (“IVSS”). The drug product is transferred to the prefilled delivery container via a delivery container adapter.

### CROSS REFERENCE TO RELATED APPLICATIONS

This application claims the benefit of U.S. Provisional Application No. 62/804,478, filed on Feb. 12, 2019, and U.S. Provisional Application No. 62/877,286, filed on Jul. 22, 2019, the entirety of each is hereby expressly incorporated herein by reference.

### FIELD OF DISCLOSURE

The present disclosure generally relates to drug delivery devices and, more particularly, to reconstitution approaches for drug delivery devices.

### BACKGROUND

Drugs are administered to treat a variety of conditions and diseases. Intravenous (“IV”) therapy is a drug dosing process that delivers drugs directly into a patient's vein using an infusion contained in a delivery container (e.g., a pliable bag). These drug dosings may be performed in a healthcare facility, or in some instances, at remote locations such as a patient's home. In certain applications, a drug product may be shipped to a healthcare facility (e.g., an inpatient facility, an outpatient facility, and/or a pharmacy) in a powdered or lyophilized form or alternatively in a liquid form.

When reconstituting these drugs for administration, it is of particular importance to maintain a sterile environment so as to not taint or otherwise damage the quality of the drug. Additionally, some classes of drugs such as bi-specific T-cell engagers may require exceptionally accurate quantities of the drug product and/or other fluids required for dosing. Oftentimes, the healthcare professional must prepare the drug by closely following a set of steps to ensure a sterile environment is maintained and that correct quantities of ingredients are added to the delivery container. As a result, the reconstitution process may be time-consuming, tedious, and may have an unacceptable or undesirable error rate.

As described in more detail below, the present disclosure sets forth systems and methods for drug delivery device reconstitution embodying advantageous alternatives to existing systems and methods, and that may address one or more of the challenges or needs mentioned herein, as well as provide other benefits and advantages.

### SUMMARY

In accordance with a first aspect, an approach for preparing a drug delivery device includes providing a prefilled delivery container and transferring a drug product contained in a drug container to the prefilled delivery container. In some examples, the drug container may be in the form of a vial, a prefilled syringe, or a cartridge. The prefilled delivery container includes a container body containing a predetermined quantity of saline solution and a predetermined quantity of IV stabilizing solution (“IVSS”). The drug product is transferred to the prefilled delivery container via a delivery container adapter.

In some examples, the drug product in the drug container is reconstituted by adding diluent to the drug container. The approach may additionally include using a vial adapter to reconstitute the drug product as well as visually inspecting the device for imperfections. In some forms, the approach may include removing the prefilled delivery container and the drug container from a common kit packaging.

In some aspects, the step of reconstituting the drug product may include providing a prefilled container having a predetermined quantity of the diluent disposed therein and transferring the diluent into the drug container. In some examples, the drug product is in the form of a half-life extended bispecific T cell engager (BiTE). In some examples, the diluent may be in the form of water for injection (“WFI”) and/or a benzyl alcohol preserved WFI.

In some examples, the vial adapter and/or the delivery container adapter may include a closed system transfer device. Further, in some examples, the IVSS may include a pretreating surfactant such as polysorbate 80.

In some forms, the predetermined quantity of saline solution may be between approximately 25 mL and approximately 500 mL per dose. The predetermined quantity of IVSS may be between approximately 1 mL and approximately 30 mL per dose. The predetermined quantity of diluent may be between approximately 0.5 mL and approximately 50 mL per dose.

In accordance with another aspect, a drug delivery system includes a prefilled delivery container including a container body and a delivery container adapter and a prefilled drug container. The container body contains a predetermined quantity of saline solution and a predetermined quantity of IV stabilizing solution (“IVSS”). The prefilled drug product includes a vial body and a vial adapter adapted to be coupled to the vial body. The vial body contains a predetermined quantity of drug product disposed therein.

### BRIEF DESCRIPTION OF THE DRAWINGS

The above needs are at least partially met through provision of the systems and approaches for drug delivery device reconstitution described in the following detailed description, particularly when studied in conjunction with the drawings, wherein:

FIG. 1 illustrates an example drug delivery system in accordance with various embodiments;

FIG. 2 illustrates an example approach for preparing a drug delivery device using the system of FIG. 1 in accordance with various embodiments; and

FIG. 3 illustrates an example usage configuration of a drug delivery system in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Turning to the figures, pursuant to these various embodiments, a drug delivery system 100 or kit and a corresponding method 200 of preparing a drug delivery device using the drug delivery system 100 are provided. The drug delivery system 100 can be used by a healthcare professional, a caregiver, or patient to prepare a drug delivery device to be delivered to a patient. The drug delivery system 100 varies from conventional systems in that a number of the components included in the system 100 come prefilled and/or premixed in correct dosage quantities. As a result, preparation of the drug delivery device by the healthcare professional, caregiver, or patient is reduced while still ensuring correct quantities of ingredients are administered. The system 100 may be used to provide intravenous, subcutaneous, intra-arterial, intramuscular, and/or epidural delivery approaches. By using the system 100, patient anxiety and or confusion may be reduced due to reduced preparation complexity and wait times caused by the drug preparation process.

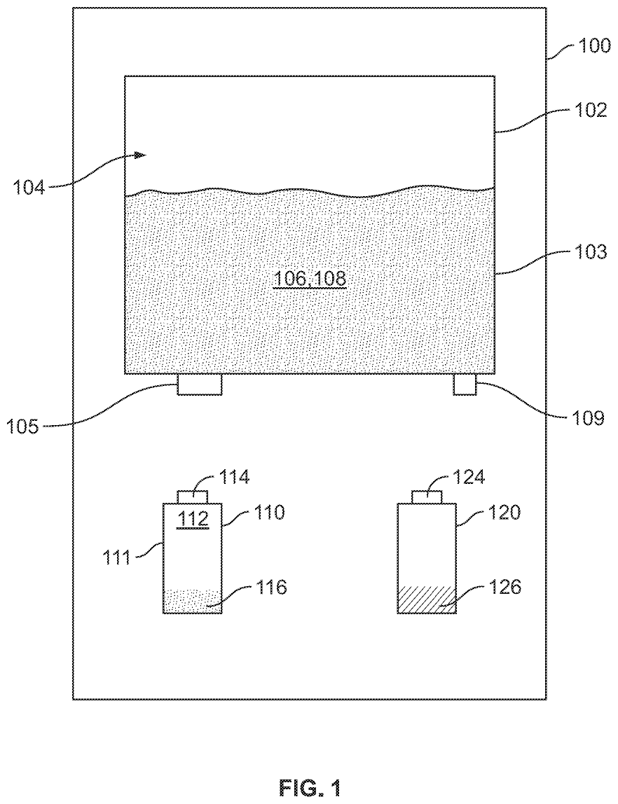

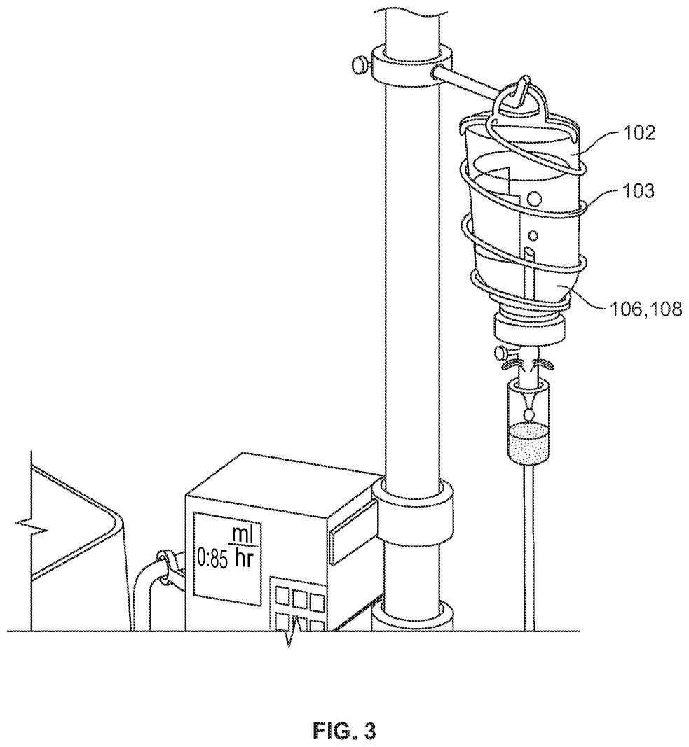

Generally, and as illustrated in FIG. 1, the drug delivery system 100 includes a prefilled delivery container 102, a prefilled drug container 110, and a prefilled reconstitution container 120. More specifically, the prefilled delivery container 102 includes a container body 103 defining an inner volume 104, a delivery container adapter 105, and an IV line outlet 109 that allows tubing to be coupled thereto in order to deliver the prescribed drug. In some examples, the prefilled delivery container 102 is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage. Other examples of suitable delivery containers are possible such as, for example, a glass bottle or container (see, e.g., FIG. 3). Example suitable prefilled delivery containers 102 are described in U.S. Appln. No. 62/804,447, filed on Feb. 12, 2019, the contents of which are incorporated by reference in their entirety.

The delivery container adapter 105 may be a closed system transfer device (“CSTD”) that allows for transfer of the drug and/or fluids into the container body 103. Example CSTD devices may include the OnGuard CSTD provided by B. Braun Medical Inc., BD PhaSeal CSTD components, Equashield CSTD, Codon CSTD, and the like. Further, non-closed system transfer devices may be used such as West Pharmaceuticals vial and bag adapters. Other examples are possible. The prefilled delivery container 102 may include any number of delivery container adapters 105 having different specifications (e.g., port sizes) to accommodate the use of different drug containers 110.

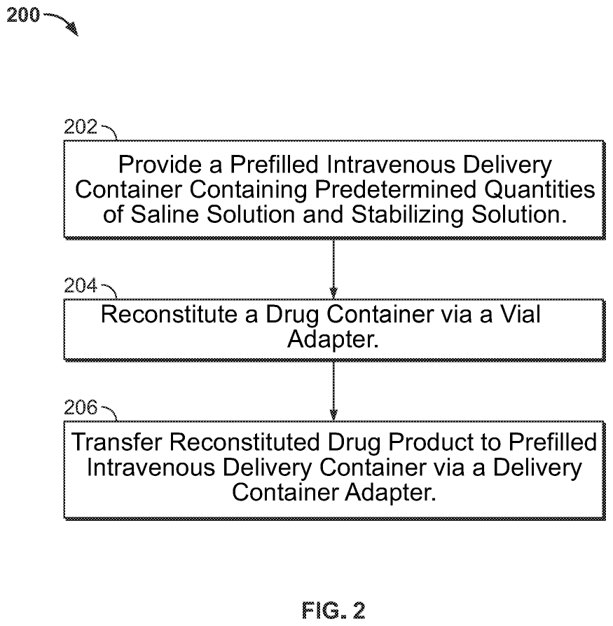

The prefilled delivery container 102 contains a predetermined quantity (e.g., a volume) of excipient solution. For example, the prefilled delivery container 102 can include a predetermined quantity of a saline solution 108 (e.g., between approximately 25 mL and 500 mL of 0.9% Sodium Chloride per dose, and preferably, approximately 110 mL or approximately 270 mL per dose, depending on the size of the container) and a predetermined quantity of an IV stabilizing solution (“IVSS”) 106. In some examples, the IVSS 106 and/or the saline solution 108 may be provided as a percentage or ratio of an overall volume of solution. In these examples, suitable quantities of IVSS 106 may range between approximately 2% and approximately 15% (e.g., between approximately 1 mL in a 50 mL container 102 and approximately 25 mL in a larger, 270 mL container per dose; see FIG. 2 at step 202). In some examples, the prefilled delivery container 102 may have a total volume of approximately 270 mL. The IVSS 106 can also act as a pretreating surfactant or a buffering component that prevents adsorption of the drug onto the walls of the container 102. For example, due to the highly potent nature of some drugs being administered, if the container 102 is not sufficiently and properly coated with the IVSS 106, it may lead to an undesirable risk of drug molecules adhering or adsorbing to the inner walls of the container 102. In the event of adsorption of the drug onto the delivery container walls 102, the dosage of the drug may be adversely impacted. In some examples, the IVSS 106 may include polysorbate 80. In some examples, the IVSS 106 formulation may include approximately 1.25 M lysine monohydrocholoride, 25 mM citric acid monohydrate, 0.1% (w/v) polysorbate 80, and has a pH of approximately 7.0. In other examples, the IVSS 106 may include similar formulations, but also have a minimum of approximately 0.9% NaCl and approximately 0.001 to approximately 0.1% (w/v) polysorbate 80. It is appreciated that different BiTEs require different final percentages of IVSS 106 in the delivery container 102. This percentage may vary between approximately 0.5% to approximately 12% of the final volume in the delivery container 102. Further, citrate may increase the risk of glass delamination if filled in glass vials. In the event that citrate is necessary for drug product stabilization (determined on a per-product basis), the delivery container 102 may be constructed from CZ or other plastic compositions. Other examples of ingredients for suitable IVSSs 106 are possible. Suitable IVSS 106 concentrations protect against protein-plastic interactions and/or surface adsorption, and more specifically, in the lower end of the concentration range where even minor losses may potentially change the effective dose. The below table illustrates example component concentrations for varying IVSS concentrations:

TABLE 1
Component Concentrations with Varying IVSS Concentrations
(top column units are (V/v) % of IVSS and saline concentration
is derived for an example using a nominal 270 mL IV-bag
volume using commercially available 0.9% saline.

| IV Bag Contents | | | | | | | | | |
|---|---|---|---|---|---|---|---|---|---|
| (IVSS Formulation | | | | | | | | | |
| Components | | | | | | | | | |
| and Final Saline | | | | | | | | | |
| Concentration) | 0.5% | 1.0% | 2.0% | 4.0% | 6.0% | 8.0% | 10.0% | 12.0% | 15.0% |
| Lysine | 0.00625 | 0.0125 | 0.025 | 0.05 | 0.075 | 0.100 | 0.125 | 0.150 | 0.188 |
| mono- | | | | | | | | | |
| hydrochloride (M) | | | | | | | | | |
| Citrate | 0.00013 | 0.00025 | 0.00050 | 0.00100 | 0.00150 | 0.00200 | 0.00250 | 0.00300 | 0.00375 |
| Monohydrate (M) | | | | | | | | | |
| Polysorbate | 0.0005 | 0.001 | 0.002 | 0.004 | 0.006 | 0.008 | 0.01 | 0.012 | 0.015 |
| 80 (% w/v) | | | | | | | | | |
| Final % of NaCL | 0.896 | 0.891 | 0.882 | 0.864 | 0.846 | 0.828 | 0.810 | 0.792 | 0.765 |
| (prepared using a | | | | | | | | | |
| stock 0.9% w/v | | | | | | | | | |
| saline solution)* | | | | | | | | | |

By providing the prefilled IVSS 106 in the delivery container 102, the overall footprint of the system 100 is reduced, as separate containers used to contain the IVSS 106 are no longer needed. Additionally, it is no longer necessary to prepare a needle and syringe assembly to inject the IVSS 106 into the delivery container, to ensure that this prepared needle and syringe assembly is sterilized, and/or to ensure a correct volume of IVSS is added to the container 102.

Some conventional systems may provide delivery containers having saline solution 108 overfill, where more saline solution 108 is provided in the delivery container 102 than what is needed for dosage. In these systems, it may be necessary to remove a volume of the saline solution 108 prior to preparing the drug dosage, which may require preparing a sterile withdrawal tool (e.g., a needle and syringe assembly) and carefully extracting an accurate amount of saline solution 108. Conversely, the disclosed system 100 additionally eliminates this process, as the delivery container 102 comes prefilled with the required quantity of saline solution 108. Additionally, the risk of a needle sticking due to the transfer of the IVSS 106 into the container 102 and/or the transfer of the saline solution 108 out of the container 102 may also be reduced or mitigated.

The prefilled drug container or syringe 110 may be in the form of a vial, a prefilled syringe, or a cartridge, and includes a vial body 111 defining an inner volume 112 and a vial adapter 114. The inner volume 112 may be sterile. In some approaches, the vial adapter 114 may also be a CSTD that mates, engages, and/or couples to the delivery container adapter 105. As with the prefilled drug delivery container 102, the inner volume 112 of the prefilled drug container 110 contains a predetermined quantity of drug product or active pharmaceutical ingredient (“API”) 116 (e.g., between approximately 2 mcg and approximately 100 mcg), depending on the BiTE and vial size, which, in the illustrated example, is in powdered form (i.e., lyophilized) requiring reconstitution. In other examples, the drug product 116 may be in liquid form and may not require reconstitution. Nonetheless, the system 100 includes an accurate quantity of drug product 116, and thus does not require the need to add additional quantities thereto in a sterile environment. In some examples, the API may be in the form of a half-life extended (“HLE”) BiTE and/or an IV-admin monoclonal antibody (“mAbs”) as desired. These HLE BiTEs include an antibody Fc region that advantageously provides different drug properties such as longer and extended half-lives. Accordingly, such APIs may be preferred due to their ability to maintain protective levels in the patient for relatively longer periods of time. Nonetheless, in other examples, the API may be in the form of a canonical-BiTE that is to be administered in a professional healthcare environment.

The prefilled reconstitution container 120 may be in the form of a vial, a prefilled syringe, or similar container that includes a reconstitution container body 121 defining an inner volume 122 and a reconstitution container adapter 124. The inner volume 122 may be sterile. In some approaches, the reconstitution container adapter 124 may also be a CSTD (or, in examples where the prefilled reconstitution container 120 is in the form of a syringe, the container adapter 124 may be a needle) that mates, engages, and/or couples to the vial adapter 114. As with the prefilled drug delivery container 102 and the prefilled drug container 110, the prefilled reconstitution container 120 contains a predetermined quantity of diluent (e.g., preservative-free water for injection or “WFI”) 126 (e.g., between approximately 0.5 mL and approximately 50 mL per dose, and more preferably, between approximately 0.5 mL and approximately 10 mL per dose) to be added to the prefilled drug container 110 for reconstitution of the drug product 116. In some examples, a benzyl alcohol preserved (or any other preservative) WFI may be used.

More specifically, the drug product 116 is reconstituted prior to addition into the delivery container 102 by mating the vial adapter 114 of the prefilled drug container 110 to the reconstitution container adapter 124 of the prefilled reconstitution container 120 and transferring the diluent 124 into the drug container 110 (see FIG. 2 at step 204). The contents may then be gently stirred, swirled, and/or inverted to mix the ingredients, thereby forming a mixed drug product. The reconstituted drug container 110 may then be visually inspected for imperfections and/or to ensure adequate mixing has occurred.

As previously noted, in some examples, the prefilled drug container 110 may be in the form of a prefilled syringe that contains the drug product 116. In these examples the drug product 116 may be in the form of a liquid BiTE formulation used in conjunction with a monoclonal antibody (mAb), In these examples, the drug product 116 may be directly added to the delivery container 102 without the use of a vial adapter system (such as the above-mentioned CSTDs) where more traditional needle-syringe injection/delivery into the container 102 is preferred, which may advantageously simplify and/or improve supply chain and manufacturing control, and may further allow for more compact commercial packaging that takes up less space in storage systems at healthcare facilities. In these examples, the prefilled drug container 110 may or may not need to be reconstituted prior to transferring the drug product 116 to the delivery container 102.

The reconstituted drug contained in the prefilled drug vial 110 may then be transferred into the drug delivery container 102 by mating the vial adapter 114 of the prefilled drug container 110 to the delivery container adapter 105 of the delivery container 102 (see FIG. 2 at step 206). As a result, this transfer of the reconstituted drug into the delivery container 102 may be performed quickly (thus greatly reducing preparation times) and safely due to the lack of withdrawal assemblies (e.g., a luer lock needle and syringe mechanism). The systems described herein avoid and/or eliminate the potential occurrence of needle sticking and/or spills due to over-pressurizing of the vial. Additionally, contamination is mitigated due to the use of closed system transfer devices, whereas conventional assemblies use components that are open to the environment and thus can be subject to contamination.

The drug delivery system 100 may include any number of additional and/or optional features or alternatives. For example, any one or ones of the delivery container adapter 105, the vial adapter 114, or the reconstitution container adapter 124 may be in the form of ports or coupling mechanisms coupled to the prefilled delivery container 102, the prefilled drug container 110, and the reconstitution container 120, respectively. These ports may in turn be coupled to a CSTD device to allow for flow between the desired containers. Accordingly, CSTD devices having suitable coupling mechanism dimensions may be included in the system 100.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/Ilia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to half-life extended BiTEs that include an antibody Fc region, BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A method of preparing a drug delivery device, the method comprising:
providing a prefilled delivery container including a container body containing: 1) a predetermined quantity of saline solution; and 2) a predetermined quantity of IV stabilizing solution (“IVSS”); and
transferring a drug product contained in a drug container to the prefilled delivery container via a delivery container adapter.

2. The method of claim 1, wherein the drug container comprises at least one of a vial, a prefilled syringe, or a cartridge.

3. The method of claim 1, further comprising the step of reconstituting the drug product in the drug container by adding diluent to the drug container, herein the step of reconstituting the drug product optionally comprises:
providing a prefilled container having a predetermined quantity of the diluent disposed therein; and
transferring the diluent into the drug container.

4. The method of claim 3, wherein the step of reconstituting the drug product comprises adding the diluent to the drug container via a vial adapter, at least one of the vial adapter or the delivery container adapter comprising a closed system transfer device.

5. (canceled)

6. (canceled)

7. (canceled)

8. The method of claim 1, wherein the IVSS comprises a pretreating surfactant or a polysorbate 80.

9. (canceled)

10. The method of claim 1, further comprising visually inspecting the drug delivery device for imperfections.

11. The method of claim 1, wherein the predetermined quantity of saline solution is between approximately 25 and approximately 500 mL per dose.

12. The method of claim 1, wherein the predetermined quantity of IVSS is between approximately 1 mL and approximately 30 mL per dose.

13. The method of claim 3, wherein the predetermined quantity of diluent is between approximately 0.5 mL and approximately 50 mL per dose or between approximately 0.5 mL and approximately 10 mL per dose.

14. (canceled)

15. The method of claim 1, further comprising the step of removing the prefilled delivery container and the drug container from a common kit packaging.

16. A drug delivery system comprising:
a prefilled delivery container including a container body and a delivery container adapter, the container body containing: 1) a predetermined quantity of saline solution; and 2) a predetermined quantity of IV stabilizing solution (“IVSS”); and
a prefilled drug container including a vial body containing a predetermined quantity of drug product disposed therein.

17. The drug delivery system of claim 16, further comprising a prefilled reconstitution container having a predetermined quantity of diluent disposed therein.

18. The drug delivery system of claim 16, wherein the prefilled drug container further includes a vial adapter to couple to the vial body, at least one of the vial adapter or the delivery container adapter comprising a closed system transfer device.

19. The drug delivery system of claim 16, wherein the drug product comprises a half-life extended bispecific T cell engager.

20. The drug delivery system of claim 16, wherein the diluent comprises at least one of water for injection (“WFI”) or a benzyl alcohol preserved WFI.

21. (canceled)

22. The drug delivery system of claim 16, wherein the IVSS comprises a pretreating surfactant.

23. The drug delivery system of claim 16, wherein the predetermined quantity of saline solution is between approximately 25 mL and approximately 500 mL per dose.

24. The drug delivery system of claim 16, wherein the predetermined quantity of IVSS is between approximately 1 mL and approximately 30 mL per dose.

25. The drug delivery system of claim 17, wherein the predetermined quantity of diluent is between approximately 0.5 mL and approximately 50 mL per dose or between approximately 0.5 mL and approximately 10 mL per dose.

26. (canceled)

27. The drug delivery system of claim 16, further comprising a kit packaging containing the prefilled delivery container and the prefilled drug container.
